(19) United States
(12) Patent Application Publication
(10) Pub. No.: US 2021/0388302 A1
(43) Pub. Date: Dec. 16, 2021

(54) COMPARTMENTALIZED CELL CULTURES FOR USAGE IN HIGH CAPACITY APPLICATIONS

(71) Applicant: CELLECTRICON AB, Mölndal (SE)

(72) Inventors: Johan PIHL, Olofstorp (SE); Mathias KARLSSON, Onsala (SE); Paul KARILA, Billdal (SE)

(21) Appl. No.: 17/282,425

(22) PCT Filed: Oct. 9, 2019
(86) PCT No.: PCT/EP2019/077380

§ 371 Date: Apr. 2, 2021

Related U.S. Application Data
(60) Provisional application No. 62/743,028, filed on Oct. 9, 2018.

(51) Int. Cl. C12M 1/00; C12M 1/32; C12M 3/06

(52) U.S. Cl. CPC C12M 23/20; C12M 23/16; C12M 23/12

(57) ABSTRACT

The disclosure relates to multi-well plates having fluidic connections between neighboring wells that are useful to produce a cell culture substrate and compliant with American National Standards Institute of the Society for Laboratory Automation and Screening (ANSI/SLAS) microplate standards.

### FIELD

The present disclosure relates to novel substrates for generation of compartmentalized cell cultures for usage in high capacity applications. Specifically, in some embodiments, the disclosure relates to an Society for Laboratory Automation and Screening (ANSI/SLAS) microplates standard compliant multi-well plate wherein groups of wells are fluidically connected to produce a cell culture substrate that can be used for a wide range of cellular assays in neurobiology research and drug discovery.

### BACKGROUND

Since its conception in the mid 1970's, compartmentalized cell cultures (CCC) have gained traction as an important methodology in neurobiology research. Campenot, R. B., Proc. Natl. Acad. Sci. U.S.A. 74: 4516-9 (1977). For example, CCC's can be used to study network communication between discrete population of neurons to study mechanisms such as synaptic communication (Vikman et al., J. Neurosci. Methods 105:175-184 (2001)), axonal transport of proteins and organelles (Bousset et al., Ann. Neurol. 72:517-524 (2013), or for the purpose of studying cell-network formation (Taylor et al., J. Neurosci. 33:5584-5589 (2013)). In addition, CCC's are being used for experiments to study cellular signaling between different cell types, for example neuron—muscle cell signaling (Zahavi et al., J. Cell Sci. 128:1241-1252 (2015)), or communication between neurons from different brain regions (Berdichevsky, Y., Staley, K. J. & Yarmush, M. L. Lab Chip 10, 999-1004 (2010).).

Traditionally, CCC's have mainly been used for basic research application where there has been limited need for high throughput or parallelization of experiments. However, because of the increasing demands from the pharmaceutical industry for more advanced and translationally relevant cell-based assay, there is now a need of being able to use CCC's in drug screening applications. For example, there is today a big interest to gain access to assay platforms that, in a relevant manner, can model prion- and prion-like mechanisms and enable screening of thousands of compounds in relatively short time frames (Zhang, M., Luo, G., Zhou, Y., Wang, S. & Zhong, Z. Phenotypic screens targeting neurodegenerative diseases. J. Biomol. Screen. 19, 1-16 (2014).). However, current state-of-the-art products cannot provide sufficient robustness or throughput to meet such demands.

To establish CCC's, cell culture substrates are being employed where discrete cell culture regions (wells) are fluidically interconnected through extremely small tubes with a diameter sufficiently large to establish a fluidic connection between the wells but sufficiently small to prevent cells to migrate between different cell-culture regions. Traditionally, CCC's were achieved through manual and very crude means: using a scalpel, grooves or scratches were manually made in the bottom of a cell culture dish. The scratches were then sealed using vacuum-grease, and discrete regions were then formed by careful positioning of a physical barrier such as glass or polytetrafluoroethylene (PTFE) rings on top of the sealed scratches. For a description of this method, see Campenot, R. B., Proc. Natl. Acad. Sci. U.S.A. 74: 4516-9 (1977). Although this method can be used to produce substrates suitable for formation of CCC's it is very cumbersome and plagued by a high failure rate. In recent years, micromachining methods have been used for production of substrates for CCC formation. For example, soft lithography and polydimethylsiloxane (PDMS, i.e. silicone rubber) casting have been employed to produce microfluidic substrates that are highly uniform and much easier to handle than the original handmade substrates. See Taylor et al., Nat. Methods 2:599-605 (2005); and Neto et al., J. Neurosci. 36:11573-11584 (2016). However, due to the rather complex microchannel networks required to enable formation of CCC's, also these microfluidic substrates display several drawbacks that prevents them for being used for efficient experimentation. For example, these substrates are difficult to fill with liquids, are prone to bubble formation, are difficult to surface modify, and are prone to delamination over time. Furthermore, because of the complex microchannel layouts required in these substrates, it is difficult to scale-up these designs into a high-density format required for high-capacity screening.

### SUMMARY

Here we present a novel substrate for production of CCC's that enable high capacity experimentation applications such as high throughput screening (HTS). The substrate is based on, but not limited to, a standard 384-well plate format wherein neighboring wells are interconnected by fluidic connections that can be made sufficiently small to prevent migration of cells, and even to maintain chemical integrity between wells. The fluidic connections have been carefully designed to enable robust liquid handling to ensure high success rates in experiments. Furthermore, the substrate can easily be surface modified using wet-chemical approaches. In order to enable usage in HTS applications, the substrate has been designed to obey all ANSI/SLAS microplate standards and is therefore compatible with most commercially available liquid handling robotics and optical readout systems available on the market.

In accordance with the description, the present disclosure encompasses, for example, a multi-well plate comprising wells, wherein at least two neighboring wells of the plate have at least one fluidic connection in the wall separating the at least two neighboring wells. In some embodiments, the multi-well plate complies with American National Standards Institute of the Society for Laboratory Automation and Screening (ANSI/SLAS) microplate standards. In some embodiments, the multi-well plate comprises a substrate produced from a thermoplastic material. In some embodiments, the thermoplastic material comprises polystyrene (PS), cyclo-olefin-copolymer (COC), cycloolefin polymer (COP), poly(methyl methacrylate (PMMA), polycarbonate (PC), polyethylene (PE), polyethylene terephthalate (PET), polyamide (Nylon®), polypropylene or polyether ether ketone (PEEK), Teflon®, PDMS, and/or thermoset polyester (TPE). In some embodiments, the multi-well plate comprises a substrate produced from cyclo-olefin-copolymer (COP), cyclo-olefin-polymer (COC) or polystyrene (PS). In other embodiments, the multi-well plate comprises a substrate produced from silicon, glass, ceramic material, or alumina. In some embodiments, the plate comprises a substrate comprising more than one layer, optionally wherein the layers are bonded by ultrasonic welding, thermocompression bonding, plasma bonding, solvent-assisted bonding, laser-assisted bonding, or adhesive bonding using glue or double adhesive tape. In some embodiments, the plate comprises a substrate coated with a protein or polymer. In some cases, the plate comprises a substrate coated with one or more of poly-1-lysine, poly-L-ornithine, collagen, laminin, Matrigel®, or bovine serum albumin. In some cases, the plate comprises a substrate comprising a surface chemically modified with one or more of poly[carboxybetaine methacrylate] (PCBMA), poly[[2-methacryloyloxy)ethyl]trimethylammonium chloride] (PMETAC), poly[poly(ethylene glycol) methyl ether methacrylate] (PPEGMA), poly[2-hydroxyethyl methacrylate] (PHEMA), poly[3-sulfopropyl methacrylate] (PSPMA), and poly[2-(methacryloyloxy)ethyl dimethyl-(3-sulfopropyl)ammonium hydroxide] (PMEDSAH).

In some embodiments, the plate further comprises at least one metallic electrode, at least one metal oxide electrode, at least one carbon electrode, and/or at least one field effect transistor detectors in wells adjacent to the fluidic connections. In some embodiments, the plate is capable of electrical read-outs comprising one or more of potential recordings, impedance spectroscopy, voltammetry and amperometry.

In some embodiments, the plate comprises at least two, at least four, at least 8, at least 16, at least 32, or at least 96 groups of three fluidically connected wells. In some embodiments, the at least one fluidic connection comprises cross-sectional dimensions of at least 0.5×0.2 mm and at most 1.0×3.0 mm, optionally with an aspect ratio ranging from 1:5 to 2:1 (height:width), In some embodiments, the at least one fluidic connection comprises cross-sectional dimensions (H and/or W) of equal to or exceeding 0.1 mm, equal to or exceeding 0.5 mm, equal to or exceeding 1 mm, equal to or exceeding 2 mm, such as dimensions ranging from 0.1×0.1 mm up to 1.0×2.0 mm (H×W), such as 0.1×0.1 mm, 0.1×0.2 mm, 0.2×0.2 mm, 0.3×0.3 mm, 0.4×0.4 mm, 0.5×0.5 mm, 0.5×1 mm, 0.6×0.6 mm, 0.7×0.7 mm, 0.8×0.8 mm, 0.9×0.9 mm, 1×1 mm, 1×1.5 mm, 1×2 mm, or 2×2 mm (H×W), or a range bounded by any of the two above dimensions. In some embodiments, the at least one fluidic connection comprises cross-sectional dimensions (H×W) of 0.1×0.1 mm to 2×2 mm, such as 0.5×0.5 mm to 1×1 mm or 0.1×0.1 mm to 1×1 mm or 0.1×0.1 mm to 0.5×0.5 mm or 0.5×0.5 mm to 2×2 mm or 0.5×0.5 mm to 1×2 mm. In some embodiments, the at least one fluidic connection comprises cross-sectional dimensions (H and/or W) between 1-20 μm, such as 1-5 μm, 1-10 μm, 5-10 μm, 10-20 μm, 10-15 μm, 15-20 μm, 5-15 μm, or comprising cross-sectional dimensions (H and/or W) of 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm, and optionally also having an aspect ratio (H×W) ranging from 1:5-2:1. In some embodiments, the fluidic connection comprises cross-sectional dimensions of equal to or less than 0.5×0.2 mm, or of 100×100 μm to 0.5×0.2 mm (H:W). In some embodiments, the fluidic connection comprises cross-sectional dimensions of equal to or less than 5×5 μm, or of 3×3 μm to 5×5 μm. In some embodiments, the dimensions, shape and number of fluidic connections are varied across the length of the at least one fluidic connection to improve neurite penetration and producibility. In some embodiments, the length of the at least one fluidic connection is at least 0.25 mm and at the most 2.0 mm. In some embodiments, the aspect ratio of the dimensions of the at least one fluidic connection ranges from 20:1 (W:H) to 1:5 (W:H).

In some embodiments, the multi-well plate comprises a 6, 12, 24, 48, 96, 384, 1536 or 3456 well format, and is optionally organized in a 2:3 rectangular matrix. In some embodiments, the multi-well plate comprises at least 2 groups of three neighboring and fluidically interconnected wells. In some embodiments, the multi-well plate comprises at least 3 groups of two neighboring and fluidically interconnected wells. In some embodiments, the multi-well plate comprises at least 1 group of four neighboring and fluidically interconnected wells.

The present disclosure also encompasses methods for high throughput screening of a material of interest, comprising screening the material of interest using the multi-well plate of any one of the embodiments herein. In some embodiments, the material of interest is a 2D cell culture. In other embodiments, the material of interest is a 3D cell culture.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides an illustration of one method of fabrication and assembly of the microplate. The two layers (layers 2 and 3) are bonded forming a laminate to seal and define the fluidic connections. The illustration shows the combined laminate (layers 2 and 3) bonded to bottomless 384-well plate (layer 1).

Layer 2: Thick, +1 mm substrate with milled through holes matching 384-well plate pattern containing trenches on underside
Layer 1: Standard 384-well plate without bottom
Bottom part (layer 3): thin transparent and preferably HCA compatible sheet

FIG. 2 illustrates the substrate containing the connections between wells, and exemplifies two possible fluidic connections between said wells.

FIG. 3 shows how different designs, i.e., pair coupled wells, three connected wells and four-connected wells can be packed in a microplate format.

FIG. 4 shows a side view of the substrate, and how this can be assembled in a three-layer as well as in a two-layer design.

The following is shown in FIG. 4A:

Layer 1: standard top-part from 384-well microtiter plate;
Layer 2: Thick, +1 mm substrate with milled through holes matching 384-well plate pattern containing trenches on underside;
Layer 3—Plate bottom, i.e. thin film (100-200 μm) bonded to above layer 2.

The following is shown in FIG. 4B:

Layer 1—standard top-part from 384-well microtiter plate
Layer 3—Plate bottom, i.e. thin film (100-200 μm) bonded to above layer 2

FIG. 5 illustrates the concept of a synaptic function assay in the substrate, and synaptic transmission and excitability and be modulated and assayed in the substrate. The following is shown in FIG. 5:

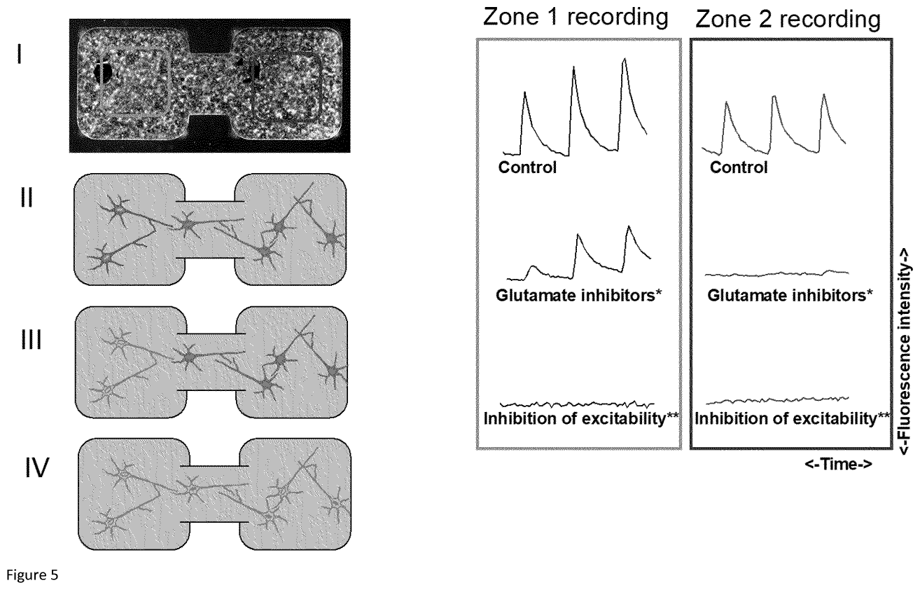

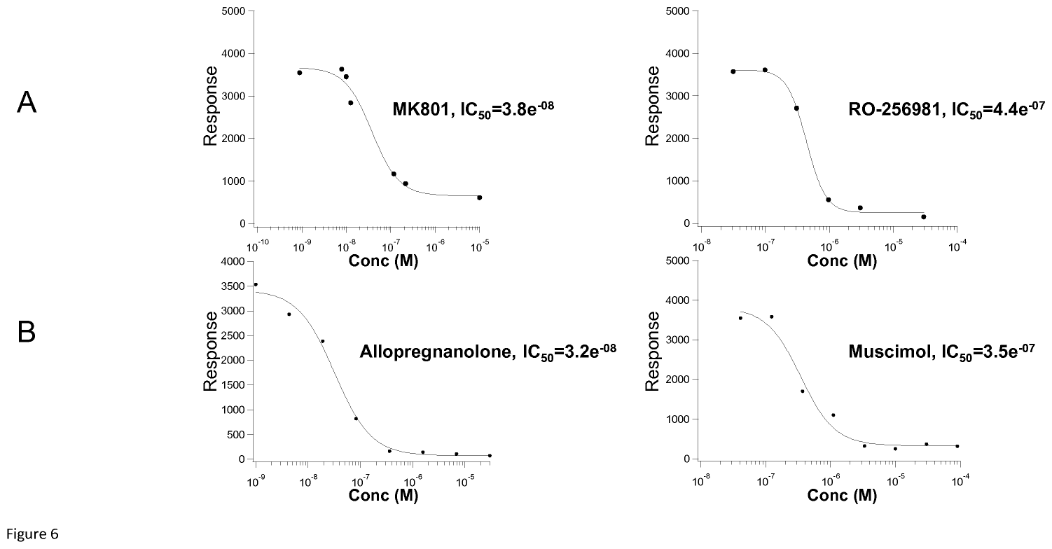

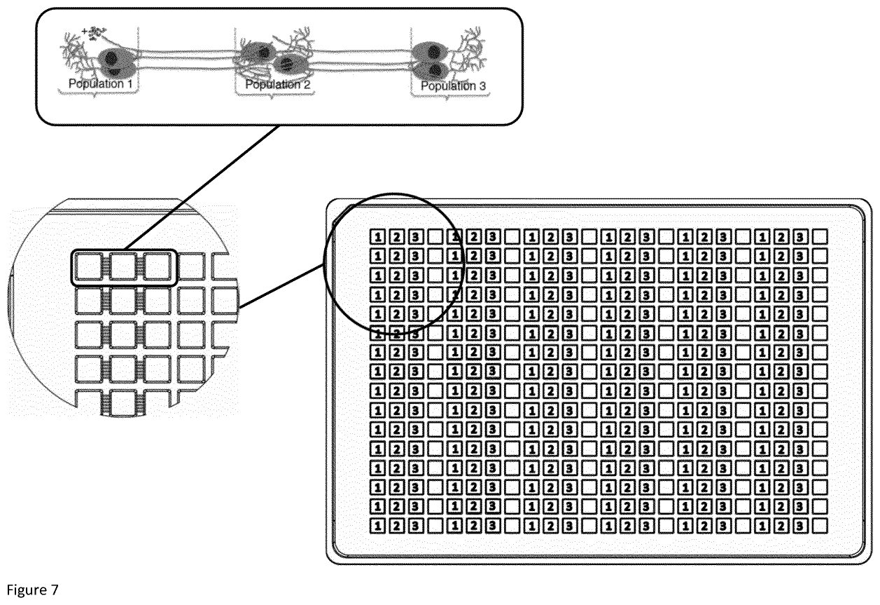

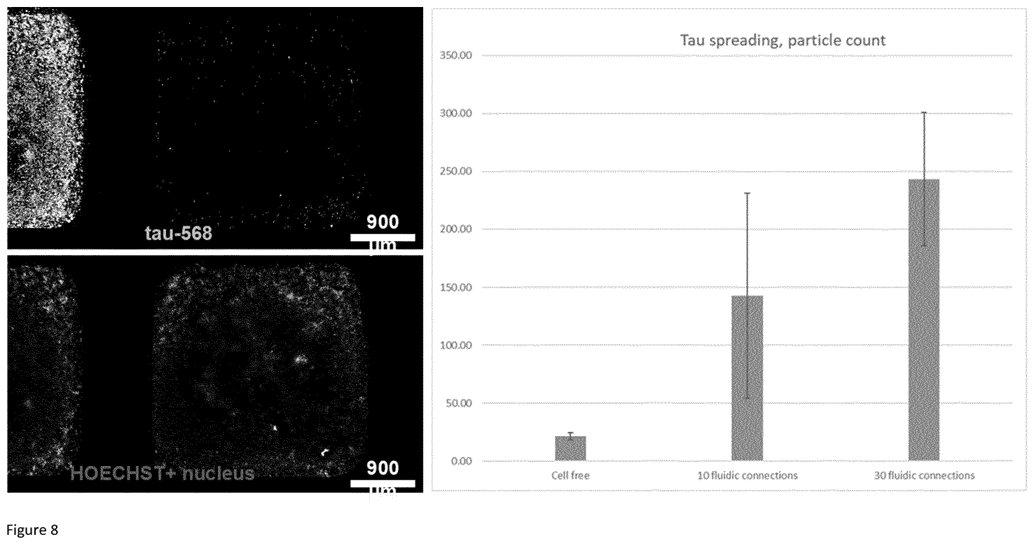

I: Micrograph of CCC with E-18 mouse cortex neurons incubated with Ca5 dye. The blue square is the stimulus zone (zone 1) and the red square is the read-out zone (zone 2);
II: Prior to electrical or chemical stimulation, the culutes are non-fluorescent;
III: Upon electrical or chemical stimulation, the cells in zone 1 fire their action potential, causing an increase in calcium fluorescence;
IV: As the action potential induced in zone 1 spreads throughout the culture via synaptically connected cells, the corresponding calcium fluorescence migrates to zone 2 where it is recorded.
Annotations in FIG. 5 are as follows:
* D-AP5 (NMDAR antagonist; 100 μM) and LY341495 (mGluR antagonist; 50 μM);
** Tetracaine (10 μM); Compound incubation: 30 min.

FIG. 6 shows example data generated from the synaptic function assay in the substrate. FIG. 6A shows Examples of NMDAR blockade, and FIG. 6B shows Examples of GABAR modulation/agonism. Electrically evoked, synaptically mediated increases in Ca2+ fluorescence can be detected. These events are mediated via the activation of AMPA and NMDARs. Pharmacological tools of known function cause predictable modulation of the observed Ca2+ signals.

FIG. 7 illustrates a prion progression and modulation assay concept. A prion-like mechanism inducer (e.g. pathogenic Tau) is added to well one, the progression of pathogenesis is then modulated in wells 2, and the modulation can be detected in well 3.

FIG. 8 shows microscopy images of spread of fluorescently labelled NDAPs (Tau particles) between cells cultures in neighboring wells connected by fluidic connections. The graph further demonstrates that spreading is dependent on the number of fluidic connections, and that cells are required for transport between wells.

### DESCRIPTION OF CERTAIN EMBODIMENTS

The present disclosure relates to a novel substrate for generation of compartmentalized cell cultures (hereinafter referred to as CCC) for usage in high capacity applications, such as HTS. Specifically, the disclosure relates to an ANSI/SLAS standard, compliant multi-well plate, which, in some embodiments can be a 384-well plate, wherein groups of wells are fluidically connected through microfabricated fluidic connections that are sufficiently small to prevent migration of cells or clusters of cells and/or to maintain chemical integrity between wells.

### Definitions

As used herein, the term “about” refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

The term “or” as used herein should be interpreted as “and/or” unless otherwise made clear from the context that only an alternative is intended.

A “multi-well plate” refers to a flat plate having wells or compartments that can be utilized as small test tubes. The multi-well plate can have, in some examples, 6, 12, 24, 48, 96, 384, 1536 or 3456 wells organized, in some embodiments, in a 2:3 rectangular matrix. A “substrate” of a plate refers to the general materials forming the plate structure and wells of the plate. A substrate can comprise one or more layers as well as one or more coatings.

A “384-well format” refers to a multi-well plate having 384 wells organized in a 2:3 rectangular matrix, i.e., 16×24 wells. In this context, the term “format” merely refers to the way in which the rows of wells are organized (e.g., 2:2, 2:3, and the number of wells in each row, that provides the total number of wells). Higher multi-well plate formats (1536-wells), for instance, having 32×48 wells or lower multi-well plate formats (96-wells) having 8×12 wells can also be used. The plate formats envisioned could be, for example, 6, 12, 24, 48, 96, 384, 1536 or 3456 wells.

The terms “connected wells,” or “interconnected wells” or “fluidically connected wells” refer to wells having direct fluidic connections between them. “Neighboring wells” refer to adjacent wells and may be interconnected by one or several fluidic connections, forming so called “groups” of wells. “Groups” of wells, as used herein, refers to wells connected directly or indirectly by fluidic connections. In some embodiments, such groups of wells may form “assayable structures” or “assayable entities” or “assayable groups,” i.e., structures or entities used for an intended assay. A group of at least 3 interconnected wells, for example, may form an assayable entity. Such groups of wells may be addressed individually or in multiple groups in parallel or sequentially on the 384 well plate.

The term “fluidic connection,” such as between wells, refers to wells having one or more connection or conduit, which depending on the purpose can allow for controlled transport or the prevention of transport of materials. In one embodiment, the fluidic connection allows transport of axons and/or dendrites but prevents transport of cells or cell bodies such as mitochondria. In this embodiment, small molecules, polymers, proteins and nanoparticles can be transported through the fluidic connections, but larger materials such as cells or mitochondria are too large to be transported and said transport can be modulated by manipulating the hydrostatic pressure. In another embodiment, the fluidic connection allows for transport of cells, clusters of cells as well as axons and dendrites. The term “cross-sectional dimensions” refers to the width and height of the fluidic connection between two wells.

The “Society for Laboratory Automation and Screening (ANSI/SLAS) microplate standards” refers to a set of standards that outlines physical dimensions and tolerances for footprint dimensions, height dimensions, outside bottom flange dimensions, well positions and well bottom elevation elevations. For example, in some embodiments, the multi-well plate complies with valid ANSI/SLAS standards, namely the ANSI/SLAS 1-2004 (R2012): Footprint Dimensions, ANSI/SLAS 2-2004 (R2012): Height Dimensions, ANSI/SLAS 3-2004 (R2012): Bottom Outside Flange Dimensions, ANSI/SLAS 4-2004 (R2012): Well Positions, and/or ANSI/SLAS 6-2012: Well Bottom Elevation.

The term “thermoplastic material” refers to a plastic material, most commonly a polymeric material, that becomes moldable or pliable above a certain temperature, and solidifies upon cooling

Compositions and Methods

a. Substrate Characteristics

In order to meet the current requirements for commercial high-throughput screening (HTS) systems, the substrate of the present disclosure, in some embodiments, may have a physical footprint and outer shape as specified in the ANSI/SLAS microplate standards. By obeying those standards, embodiments of the present disclosure may be compatible with established robotic plate handling systems, liquid handling systems, and optical readout systems utilized in HTS. However, as standards for microplates are subject to future changes in shape or design, the present invention is also compatible with a variety of shapes and sizes of multi-well plates.

In one embodiment of the disclosure, the substrate is composed of three parts:

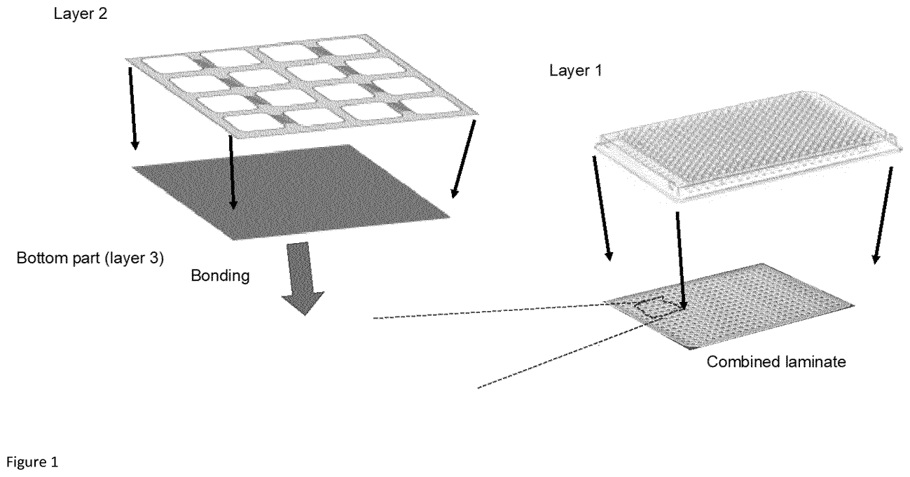

First, the substrate is composed a top part that defines the outer dimensions and shape of the substrate. Also, the first part defines the macroscopic part of the wells or cell culture regions. The size and geometry of these wells should be designed to facilitate liquid handling and cell-culture processes. In one embodiment of the present disclosure, a 384-well format is used. However, in other embodiments of the disclosure, multi-well plates having 6, 12, 24, 48, 96, 1536 or 3456 wells can be used. (FIG. 1). For example, in embodiments that use a 384 well plate, the plate comprises at least 96 groups of three neighboring and fluidically interconnected wells, at least 192 groups of two neighboring and fluidically interconnected wells, or at least 96 groups of four neighboring and fluidically interconnected wells. Multi-well plates with 6, 12, 24, 48, 96, 1536 or 3456 wells having groups of two or three interconnected wells could also be used.

The second and middle part of the substrate defines the fluidic connections between neighboring wells. (FIG. 1) Depending on the application, the size and length of these fluidic connections can be varied and depends on the type of cell-based assay where the substrate is to be used. For example, for synaptic efficacy assays, focus is on creating cell cultures where local chemical integrity can be maintained to enable induction of a local chemical stimulus in the cell-culture. In this embodiment of the disclosure, fluidic connections can be used that have cross sectional diameters equal to or exceeding 0.1 mm, equal to or exceeding 0.5 mm, equal to or exceeding 1 mm, equal to or exceeding 2 mm, such as dimensions ranging from 0.1×0.1 mm up to 1.0×2.0 mm (H×W), such as 0.1×0.1 mm, 0.1×0.2 mm, 0.2×0.2 mm, 0.3×0.3 mm, 0.4×0.4 mm, 0.5×0.5 mm, 0.5×1 mm, 0.6×0.6 mm, 0.7×0.7 mm, 0.8×0.8 mm, 0.9×0.9 mm, 1×1 mm, 1×1.5 mm, 1×2 mm, or 2×2 mm (H×W), or a range bounded by any of the two above dimensions. In some embodiments, the dimensions may range from 0.1×0.1 mm to 2×2 mm, such as 0.5×0.5 mm to 1×1 mm or 0.1×0.1 mm to 1×1 mm or 0.1×0.1 mm to 0.5×0.5 mm or 0.5×0.5 mm to 2×2 mm or 0.5×0.5 mm to 1×2 mm, for example, having an aspect ratio (H×W) ranging from 1:5-2.

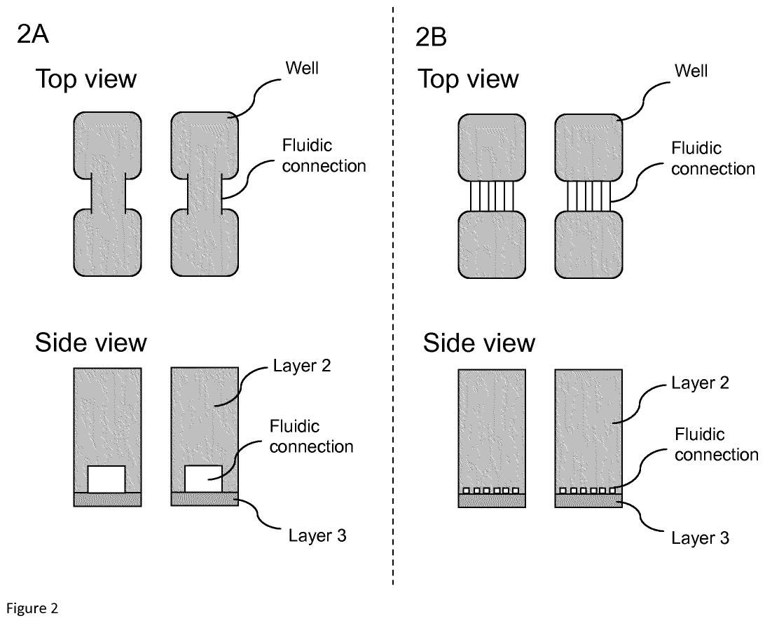

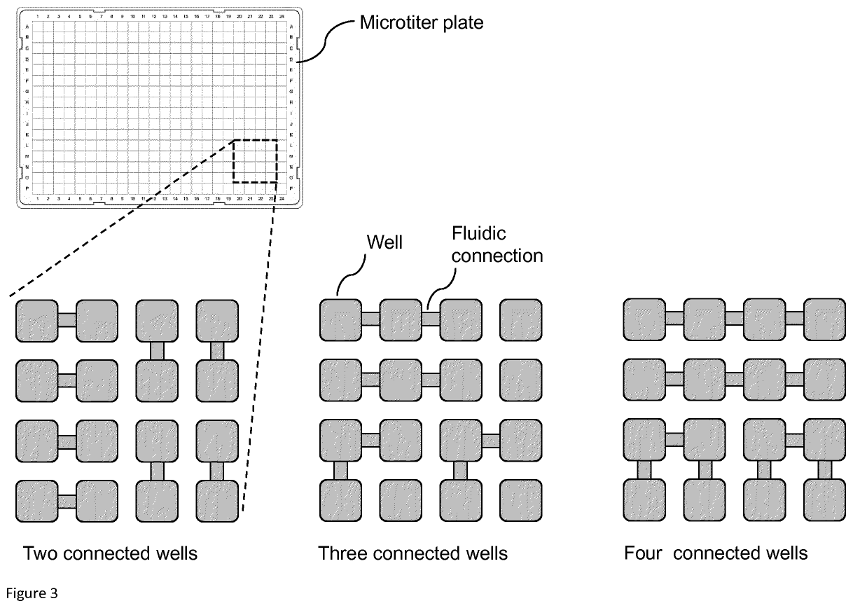

For assaying prion-like mechanisms, much smaller connections may be used, for example, to prevent migration of cells between different cell-culture zones (wells). (FIG. 2). The cross-sectional dimensions of the fluidic connections may in this case comprise one or more connections having a dimension between 1-20 μm, such as 1-5 μm, 1-10 μm, 5-10 μm, 10-20 μm, 10-15 μm, 15-20 μm, 5-15 μm, or having a dimension (H or W) of 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm, and optionally also having an aspect ratio (H×W) ranging from 1:5-2:1. In some embodiments the cross-sectional dimensions of the fluidic connections comprise at least 1×0.5 mm.

In some embodiments the cross-sectional dimensions of the fluidic connections comprise less than 1×0.5 mm. In one embodiment, the fluidic connection is comprised of one or more connections having dimensions as small as 3×3 μm. The connections could also have larger dimensions, up to 100×100 μm. The aspect ratio, i.e., the ratio between width and height of cross-sectional dimensions could range from aspect ratios of 20:1 (W:H) to 1:5 (W:H), such as from 20:1 to 10:1, from 10:1 to 5:1, from 5:1 to 1:1, from 2:1 to 1:2, from 1:1 to 1:2, from 1:1 to 1:5, or from 1:2 to 1:5 (all W:H).

The shape and size of the fluidic connections may vary across the length-axis of the connection to optimize parameters such as producibility, fluid wetting and filling, and entrance of cellular processes into the fluidic connectors. For example, incorporation of funnel-like structures at the entrances of the fluidic connections can improve neurite guiding and penetration and varying the height of the fluidic connections can improve mechanical stability and thus producibility. In one embodiment, channels having 6×8 μm (W×H) dimensions are expanded to 20×8 μm (W×H) over a distance of 200 μm, thereby improving axon and dendrite guidance into the fluidic connection. In another embodiment, these funnel-like structures at the are joined together to form one large fluidic connection at the entrance, further improving neurite guidance and penetration. In one embodiment, this large fluidic connection at the entrance is also higher, significantly improving production yield of the multi-well plate. In one embodiment, the height of the fluidic connection is increased from 8 μm to 50 μm, but other heights can also be envisioned.

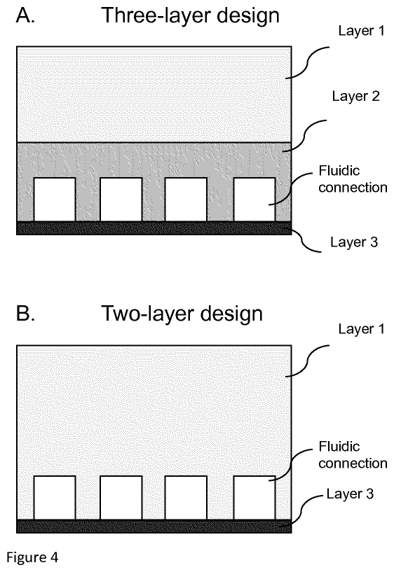

Also, depending on the assay application, the number of connected wells may vary. In one embodiment of the disclosure, the substrate contains several units of pair-coupled wells (i.e., two connected walls), in a second embodiment of the disclosure the substrate contains several units of three connected wells, and in a third embodiment of the disclosure, the substrate contains several units of four or more connected wells. (FIG. 3) In one embodiment of the disclosure, the fluidic connections are formed directly in the first layer of the substrate thus completely omitting the need to include a second layer in the substrate. (FIG. 4).

The third part of the substrate defines the bottom of the substrate. In order to enable high resolution imaging readouts, in some embodiments this bottom part of the substrate is optically transparent within the visible and far UV light spectra range and sufficiently thin to enable imaging using high numerical aperture microscope objectives. Accordingly, in some embodiments, the thickness of the third bottom part is less than 200 μm, such as 10-50 μm, 50-100 μm, or 100-200 μm. In other embodiments of the disclosure where high-resolution imaging is not utilized, the bottom layer of the substrate can be made thicker to increase mechanical robustness of the substrate. (FIG. 1). Accordingly, in some embodiments, the thickness of the third bottom part is in the range of 200-1000 μm, such as 200-500 μm, or 300-700 μm, or 500-1000 μm, or 200 μm, 300 μm, 400 μm, 500 μm, 600 μm, 700 μm, 800 μm, 900 μm, or 1000 μm.

In some embodiments, the substrate may be equipped with additional parts, such as metallic electrode, metal oxide electrodes, carbon electrodes, or field effect transistor detectors in the wells adjacent to the fluidic connectors to enable electrical read-outs including but not limited to filed potential recordings, impedance spectroscopy, or voltammetry and amperometry.

B. Methods of Substrate Production

The substrate of the present disclosure can be produced from a wide range of materials such as thermoplastics. Exemplary thermoplastic materials may include, for example, polystyrene (PS), cyclo-olefin-copolymer (COC) or cycloolefin polymer (COP), poly(methyl methacrylate (PMMA), polycarbonate (PC), polyethylene (PE), polyethylene terephthalate (PET), polyamide (Nylon®), polypropylene or polyether ether ketone (PEEK) Additional material groups may include perfluorinated materials like Teflon®, silicone polymers like PDMS, thermoset polymers such as thermoset polyester (TPE) or hard crystalline or amorphous materials, such as silicon, glass or ceramics such as alumina. However, to meet cost criteria for high-throughput screening where disposable substrates may be preferred, and large volumes of substrates may be consumed, the substrate may be produced from PS, COC or COP, as these materials may be amenable to cost-efficient high-volume production methods such as injection molding, hot embossing, or computer-aided manufacturing (CAM) micro machining. In some embodiments, the material to be used in the substrate is amenable for surface coatings to enable culture of cells. For example, it may be desirable in some embodiments to carry out physical surface treatments, e.g. plasma treatment or corona discharge, as well as to coat the substrate with materials proteins or polymeric materials such as poly-1-lysine, poly-L-ornithine, collagen, laminin, Matrigel®, bovine serum albumin or other protein solutions. Furthermore, chemical modifications can also be grafted onto the surface, in example poly[carboxybetaine methacrylate] (PCBMA), poly[[2-methacryloyloxy)ethyl]trimethylammonium chloride] (PMETAC), poly[poly(ethylene glycol) methyl ether methacrylate] (PPEGMA), poly[2-hydroxyethyl methacrylate] (PHEMA), poly[3-sulfopropyl methacrylate] (PSPMA), and poly[2-(methacryloyloxy)ethyl dimethyl-(3-sulfopropyl)ammonium hydroxide] (PMEDSAH).

To assemble the different layers of the substrate, a range of bonding methods can be utilized. For example, methods like ultrasonic welding, thermocompression bonding, plasma bonding, solvent-assisted bonding, laser-assisted bonding or adhesive bonding using glue or double adhesive tape can be used. Although not preferred from a manufacturing viewpoint, the substrate may be composed of different materials. In one embodiment of the disclosure, the bottom layer is composed of glass whereas the other layers are composed of thermoplastics or a silicone polymer material.

### EXAMPLES

### Example 1—Assay to Study Synaptic Efficacy

Subtle changes in the environment of complex neuronal networks may cause either breakdown or creation of synaptic connections. Drug discovery screening for neurological and psychiatric diseases would thus benefit from robust, automated, quantitative in vitro assays that monitor changes in neuronal function. A synaptic function assay should yield data relevant to therapeutic areas, particularly in relation to neurodegenerative disorders, and should also provide evidence that compounds of interest engage with native targets to produce changes in neuronal function. The low throughput of conventional electrophysiological techniques means only a small number of compounds can be tested over a realistic time frame for a drug discovery project.

In this example, we demonstrate how the cell-culture substrate of the present disclosure can be used in combination with an optical electrophysiology platform to enable screening of larger compound sets and will allow researchers to be more confident that they have selected the correct compound(s) and concentration(s) before moving forwards to utilizing standard electrophysiological techniques in intact neuronal tissue.

For establishment of a synaptic function assay, the main purpose of utilizing a CCC is to create at least two chemically and electrically discrete zones in a cell-culture. The first zone will be used for induction of the cellular action potential, and the second zone is utilized as a read-out zone to monitor whether the action potential from zone 1 has propagated to zone 2 through synaptically connected cells. Thus, the purpose of the CCC substrate is to form zone 1 and zone 2 in the cell-culture, and to ensure these zones can maintain chemical and electrical integrity.

To achieve this, embryonic day 18 (E-18) mouse cortical tissue was dissociated mechanically, and the single cell solution was plated in a cell-culture substrate having a 384-well plate format containing 192 pair coupled wells where the fluidic connection consisted of one, large connection with cross-sectional dimensions of 0.2×2.0 mm. Prior to seeding the cells, the substrate was coated first with a 0.01% poly-L-ornithine solution overnight at 37° C. The wells were thereafter washed with PBS with Ca²⁺/Mg²⁺ after which laminin diluted to 10 μg/ml in PBS with Ca²⁺/Mg²⁺ was added and incubated for 2 h at 37° C. Laminin was removed just prior to cell seeding. After 14 days in culture and on the day of the experiment, the cells in the substrate were loaded with a calcium indicator followed by acute incubation (1 hour) with compounds of interest in concentration response-format.

To induce the action potential in zone 1 of the substrate, the plate was placed in a dynamic fluorescence imaging plate reader (Cellaxess Elektra®, Cellectricon AB, Molndal, Sweden) capable of parallel monitoring the calcium fluorescence in all wells in the plates. In this example, a capillary electrode array was used (Cellaxess Elektra® Electrostimulation Module, Cellectricon AB, Molndal, Sweden)) to provide parallel and homogeneous external electrical fields to all zone 1's in the plate. Alternatively, an elevated concentration of potassium (typically 25-100 mM supplied in a osmolarity-adjusted solution), or other action potential activating agents such as veratridine was added to zone 1's to induce the action potential. Synaptic transmission was assessed by analyzing calcium fluorescence transients in zone 2's of the plate. It was also possible to monitor neuronal excitability in the cell-culture by studying the electrically, or chemically, evoked calcium transients in zone 1's using the above experimental protocol. The assay concept is illustrated in detail in FIG. 5.

Methods:

Briefly, the dissection of mouse cortices was performed under sterile conditions. After dissection, Eppendorf tubes were placed and kept in an ice-filled, insulated container throughout the dissection until preparation and cell seeding. The mouse cortical preparations were performed in the cell laboratory at the applicant, Cellectricon, under sterile conditions. The tissue was transferred from the original vials with a minimal amount of medium (Hibernate E minus Ca²⁺ BrainBits LLC, Springfield, Ill., USA) to tubes pre-filled with trypsin 0.05%+EDTA in Hibernate E using a fire polished large bore size Pasteur pipette. The tissue was incubated in a 37° C. water bath for 15 minutes. The trypsin+EDTA solution was thereafter removed and Hibernate E supplemented with 10% fetal bovine serum added. The tissue was gently triturated with a sterile 9″ silanized glass Pasteur pipette to dissociate the tissue. The solution was left for 1 minute in order for the non-dissociated tissue to precipitate. The supernatant from each tube was then transferred and pooled in a tube. To each remaining pellet, fresh Hibernate E minus Ca²⁺ was added. The trituration procedure above was repeated, and the cell suspension transferred to the cell suspension tube. After the final trituration, the cell suspension was divided into two separate tubes and centrifuged for 5 min at 250×g at room temperature. The supernatant in each tube was removed, and the pellet was carefully re-suspended by sequential addition of NbActiv4 (BrainBits LLC, Springfield, Ill., USA) in one tube, and DMEM in one tube. The cell suspensions were carefully triturated between each addition to dissociate cell aggregates. The cell suspension was strained on a 40 μm pore diameter cell strainer to reduce the amount of large cell clusters. Appropriate medium was added to each cell suspension to yield a total of 3 ml and cells were counted using a Scepter cell counter (Scepter® Cell counter 2.0, Merck Millipore, according to manufacturer's manual). Cell suspension was diluted to 1 000 000 cells/ml and 50 μl cell suspension was added per well into a 384-well plate. In all experiments, plates were incubated at 37° C., 5% CO₂, 95% humidity for 13-15 days. To support viability of cells and nutrient supply, 50% medium was changed on day 3, and subsequent half media exchanges were performed in intervals every 3 to 4 days.

EFS and Calcium imaging experiments were carried out after 14 DIV. These experiments were performed on the Cellaxess Elektra® platform (Cellectricon AB, Molndal, Sweden), equipped with an imaging module. The temperature in the instrument was kept at 31-32° C. during the experiment. At the day of the experiment, the calcium indicator Calcium 5 was dissolved either in NbActiv4 (mouse cortical neurons) or complete medium (human iPSC neurons). Cell cultures were stained with Calcium 5 (resulting in 10% medium exchange). The cells were then incubated at 37° C., 5% CO₂. Approximately 1 h after Calcium 5 addition the cell plate was inserted in the Cellaxess Elektra® and spontaneous neuronal activity were measured as alterations of calcium signal over time. Afterwards, a series of electrical field stimulation were applied in zone's 1 in the multi-well plates. The response to the stimuli was simultaneously monitored as a change in calcium intensity (camera image acquisition frequency was set to 20 Hz with 39 ms exposure/image with the camera binned 4×4. Differences in the calcium response ratio (peak response/baseline level) were then used to determine % effect. Using this assay, we have characterized a diverse array of chemical agents that modulate synaptic function to produce pharmacological data. For example, concentration response data for compounds targeting a number of different mechanisms have been carried out. Examples include NMDR receptor antagonists and GABAAR receptor modulator. In most instances, the data from our synaptic function assay agrees well with literature data. The two top graphs in FIG. 6 outline concentration response data for compounds which block synaptic transmission through inhibition of the NMDA receptor, the receptor of main importance for propagation of signal within the synapse. The two bottom graphs in FIG. 6 outlines how synaptic transmission can be blocked through positive modulation of the GABAA receptor, the main inhibitory receptor in the synapse. In both cases the result correlates well with existing literature data. Furthermore, the assay can easily be scaled to a format that maintains a medium level of throughput (for example, less than 20,000 compounds) thus being useful for screening of, for example, focused HTS libraries. This could, for example, be accomplished by utilizing a 384-well format multi-well plate having 192 groups. Using this format, a library of 20,000 compounds could be screened in duplicate in less than three working weeks, assuming 15% added experimental controls, 10% re-screened plates, at a screening pace of 10 plates/day.

There is a need for innovative, functional screening approaches to address disease mechanisms for complex, multi-factorial psychiatric and neurological disorders. In this regard, the application of the present disclosure in combination with cellular models of CNS disease may lead to the discovery of novel compounds or targets that restore aberrant synaptic function and serve as the basis for new mechanism-based treatments.

### Example 2—Assay to Study Spreading of Neurodegenerative Disease Associated Peptides (NDAP) in Neuronal Circuits

Spreading of neurodegenerative disease associated peptides (NDAPs) within the brain is considered as one of the major pathological mechanisms in progressive neurodegenerative diseases, such as Alzheimer's and Parkinson's disease. In this concept, pathological soluble forms of NDAPs, such as amyloid-beta, alpha-synuclein and tau proteins, are incorporated by neurons where they cause progression of protein misfolding, synapse elimination and neuronal cell loss. Moreover, a plethora of literature reports a prion-like mechanism of intracellular NDAPs, i.e. the intracellular transport of NDAPs and spreading from one neuron to another. Since neurodegenerative diseases are still virtually non-treatable, a high throughput assay platform that reflect all these complex neuropathological features in vitro and that allows screening and profiling of larger compound sets to prevent this neurodegenerative cascade, represents an urgent unmet clinical need.

Using the substrate of the present disclosure, we have been able to create a unique high throughput in vitro assay that reflects all hallmarks of the neurodegenerative disease cascade within CNS neuronal circuits. Mouse cerebral cortical neuronal cultures are being used since neurons in these cultures develop extensive processes and form functional synaptic connections in vitro.

In detail, mouse cortical E18 neurons were plated in a customized CCC substrate having a 384-well plate format containing 96 experimental units composed of three neighboring wells that were fluidically connected. In this application, it is of paramount importance that cells cannot migrate between neighboring wells, and therefore the fluidic connections were made smaller than a neuronal cell body. Therefore, each fluidic connection consisted of 10-30 holes with cross sectional diameters of 6×8 μm. Prior to seeding the cells, the substrate was coated first with a 0.01% poly-L-ornithine solution overnight at 37° C. The wells were thereafter washed with PBS with Ca²⁺/Mg²⁺ after which laminin diluted to 10 μg/ml in PBS with Ca²⁺/Mg²⁺ was added and incubated for 2 h at 37° C. Laminin was removed just prior to cell seeding. Cells were then prepared and cultured as described in Example 1. After 7 days in culture, the cells in zone 1's in all experimental units in the plate were treated with a 50 nM solution of NDAP polymers, e.g. patient-derived material such as pathogenic Tau protein oligomers extracted from the CSF of Alzheimer's patient. The plate was thereafter brought back into the incubator and the cells were cultured for 7 more days. After 14 DIV, the cells in the plate were fixed and stained for neuronal and assay-specific markers using immunocytochemical protocols, and high-content imaging is used to describe NDAP uptake, intraneuronal spreading and NDAP-mediated alteration of synapses and neuronal survival. Briefly, cells were fixed using 4% PFA in PBS or methanol. Neurons were evaluated using antibodies binding to mouse MAP-2AB (1:1000), chicken MAP-2AB (1:10000) or bTubIII (1:1000). Hoechst (nuclei) staining was also included. Anti-bTubIII (Sigma-Aldrich Sweden AB, Stockholm, Sweden) (1:1000), -PSD-95 (1:1000), -Synaptophysin (1:1000), -tau (1:1000), respectively, were combined with MAP-2AB antibodies (Sigma-Aldrich Sweden AB, Stockholm, Sweden). High-content imaging (HCA) analysis was performed using an Operetta® high content imager at 10×, 20× or 40× magnification (PerkinElmer).

To screen for modulators of spreading of NDAPs across synaptically coupled neurons, chemical, biologics- or genetic intervention can be performed in well two of the experimental unit to modulate the cell cultures ability to spread NDAPs, and well three of the unit is used to measure presence of NDAPs intracellularly that have spread through the cell-culture from well 1. An illustration of the assay concept is shown in image 7. Using this assay concept, we have demonstrated uptake and modulation of NDAPs. After 7 DIV, pathological Tau extracted from CSF of human AD patients was added as per above to the cells in zone's 1 in all experimental units. By balancing the liquid levels between the wells in the experimental units, it was ensured that no mass transport of Tau material took place between the wells in the experimental units. The pathological Tau was rapidly taken up by the cultures, and after 9 DIV, a modulating antibody was added to zone's 2 in all experimental units with the aim of modulating propagation of the Tau pathology in the culture. Again, liquid levels were balanced to ensure that no mass transport of antibody material took place between the wells in the experimental units. At 14-16 DIV, synaptic function was assessed in zone 3's in all experimental units in the plate by analyzing calcium fluorescence transients. Following this, cultures were fixed and stained for Beta tubulin type 3 and endogenous Tau (MAPT) and high-resolution images were acquired using a high content imager. Effects on synaptic function of the cultures together with effects on network integrity and endogenous Tau levels as analyzed by automated image analysis enabled high capacity screening for modulators of Tauopathy progression.

To our knowledge, this approach will show sufficient capacity and robustness to allow screening and profiling of larger compound sets in the search for molecules preventing spreading of NDAPs across synaptically coupled neurons.

### REFERENCES

1. Campenot, R. B. Local control of neurite development by nerve growth factor. Proc. Natl. Acad. Sci. U.S.A. 74, 4516-9 (1977).
2. Vikman, K. S., Backström, E., Kristensson, K. & Hill, R. H. A two-compartment in vitro model for studies of modulation of nociceptive transmission. J. Neurosci. Methods 105, 175-184 (2001).
3. Bousset, L., Sourigues, Y., Covert, M. & Melki, R. Neuron-to-neuron transmission of α-synuclein fibrils through axonal transport. Ann. Neurol. 72, 517-524 (2013).
4. Taylor, A. M., Wu, J., Tai, H.-C. & Schuman, E. M. Axonal Translation of Catenin Regulates Synaptic Vesicle Dynamics. J. Neurosci. 33, 5584-5589 (2013).
5. Zahavi, E. E. et al. A compartmentalized microfluidic neuromuscular co-culture system reveals spatial aspects of GDNF functions. J. Cell Sci. 128, 1241-1252 (2015).
6. Berdichevsky, Y., Staley, K. J. & Yarmush, M. L. Lab Chip 10, 999-1004 (2010).).—Communication between different brain regions
7. Zhang, M., Luo, G., Zhou, Y., Wang, S. & Zhong, Z. J. Biomol. Screen. 19, 1-16 (2014—Need for HTS prion-like mechanism platform
8. Anne M Taylor, Mathew Blurton-Jones, Seog Woo Rhee, David H Cribbs, Carl W Cotman, and N. L. J. A microfluidic culture platform for CNS axonal injury, regeneration and transport. Nat. Methods 2, 599-605 (2005).
9. Neto, E. et al. Compartmentalized Microfluidic Platforms: The Unrivaled Breakthrough of In Vitro Tools for Neurobiological Research. J. Neurosci. 36, 11573-11584 (2016).
10. ANSI/SLAS 1-2004 (R2012): Footprint Dimensions
11. ANSI/SLAS 2-2004 (R2012): Height Dimensions
12. ANSI/SLAS 3-2004 (R2012): Bottom Outside Flange Dimensions
13. ANSI/SLAS 4-2004 (R2012): Well Positions
14. ANSI/SLAS 6-2012: Well Bottom Elevation

The foregoing written specification is considered to be sufficient to enable one skilled in the art to practice the embodiments. The foregoing description and Examples detail certain embodiments. It will be appreciated, however, that no matter how detailed the foregoing may appear in text, the embodiments may be practiced in many ways and should be construed in accordance with the appended claims and any equivalents thereof.

1. A multi-well plate comprising wells, wherein at least two neighboring wells of the plate have at least one fluidic connection in the wall separating the at least two neighboring wells.

2. The multi-well plate of claim 1, wherein the multi-well plate complies with American National Standards Institute of the Society for Laboratory Automation and Screening (ANSI/SLAS) microplate standards.

3. The multi-well plate of claim 1, wherein the multi-well plate comprises a substrate produced from a thermoplastic material.

4. The multi-well plate of claim 3, wherein the thermoplastic material comprises polystyrene (PS), cyclo-olefin-copolymer (COC), cycloolefin polymer (COP), poly(methyl methacrylate (PMMA), polycarbonate (PC), polyethylene (PE), polyethylene terephthalate (PET), polyamide (Nylon®), polypropylene or polyether ether ketone (PEEK), Teflon®, PDMS, and/or thermoset polyester (TPE).

5. The multi-well plate of claim 1, wherein the multi-well plate comprises a substrate produced from cyclo-olefin-copolymer (COP), cyclo-olefin-polymer (COC) or polystyrene (PS).

6. The multi-well plate of claim 1, wherein the multi-well plate comprises a substrate produced from silicon, glass, ceramic material, or alumina.

7. The multi-well plate of claim 1, wherein the plate comprises a substrate comprising more than one layer, optionally wherein the layers are bonded by ultrasonic welding, thermocompression bonding, plasma bonding, solvent-assisted bonding, laser-assisted bonding, or adhesive bonding using glue or double adhesive tape.

8. The multi-well plate of claim 1, wherein the plate comprises a substrate coated with a protein or polymer.

9. The multi-well plate of claim 8, wherein the plate comprises a substrate coated with one or more of poly-l-lysine, poly-L-ornithine, collagen, laminin, Matrigel®, or bovine serum albumin.

10. The multi-well plate of claim 1, wherein the plate comprises a substrate comprising a surface chemically modified with one or more of poly[carboxybetaine methacrylate] (PCBMA), poly[[2-methacryloyloxy)ethyl]trimethylammonium chloride] (PMETAC), poly[poly(ethylene glycol) methyl ether methacrylate] (PPEGMA), poly[2-hydroxyethyl methacrylate] (PHEMA), poly[3-sulfopropyl methacrylate] (PSPMA), and poly[2-(methacryloyloxy)ethyl dimethyl-(3-sulfopropyl)ammonium hydroxide] (PMEDSAH).

11. The multi-well plate of claim 1, wherein the plate further comprises at least one metallic electrode, at least one metal oxide electrode, at least one carbon electrode, and/or at least one field effect transistor detectors in wells adjacent to the fluidic connections.

12. The multi-well plate of claim 11, wherein the plate is capable of electrical read-outs comprising one or more of potential recordings, impedance spectroscopy, voltammetry and amperometry.

13. The multi-well plate of claim 1, wherein the plate comprises at least two, at least four, at least 8, at least 16, at least 32, or at least 96 groups of three fluidically connected wells.

14. The multi-well plate of claim 1, wherein the at least one fluidic connection comprises cross-sectional dimensions of at least 0.5×0.2 mm and at most 1.0×3.0 mm, optionally with an aspect ratio ranging from 1:5 to 2:1 (height:width).

15. The multi-well plate of claim 1, wherein the at least one fluidic connection comprises cross-sectional dimensions (H and/or W) of equal to or exceeding 0.1 mm, equal to or exceeding 0.5 mm, equal to or exceeding 1 mm, equal to or exceeding 2 mm, such as dimensions ranging from 0.1×0.1 mm up to 1.0×2.0 mm (H×W), such as 0.1×0.1 mm, 0.1×0.2 mm, 0.2×0.2 mm, 0.3×0.3 mm, 0.4×0.4 mm, 0.5×0.5 mm, 0.5×1 mm, 0.6×0.6 mm, 0.7×0.7 mm, 0.8×0.8 mm, 0.9×0.9 mm, 1×1 mm, 1×1.5 mm, 1×2 mm, or 2×2 mm (H×W), or a range bounded by any of the two above dimensions.

16. The multi-well plate of claim 1, wherein the at least one fluidic connection comprises cross-sectional dimensions (H×W) of 0.1×0.1 mm to 2×2 mm, such as 0.5×0.5 mm to 1×1 mm or 0.1×0.1 mm to 1×1 mm or 0.1×0.1 mm to 0.5×0.5 mm or 0.5×0.5 mm to 2×2 mm or 0.5×0.5 mm to 1×2 mm.

17. The multi-well plate of claim 1, wherein the at least one fluidic connection comprises cross-sectional dimensions (H and/or W) between 1-20 μm, such as 1-5 μm, 1-10 μm, 5-10 μm, 10-20 μm, 10-15 μm, 15-20 μm, 5-15 μm, or comprising cross-sectional dimensions (H and/or W) of 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm, and optionally also having an aspect ratio (H×W) ranging from 1:5-2:1.

18. The multi-well plate of claim 1, wherein the fluidic connection comprises cross-sectional dimensions of equal to or less than 0.5×0.2 mm, or of 100×100 μm to 0.5×0.2 mm (H:W).

19. The multi-well plate of claim 1, wherein the fluidic connection comprises cross-sectional dimensions of equal to or less than 5×5 μm, or of 3×3 μm to 5×5 μm.

20. The multi-well plate of claim 17, where the dimensions, shape and number of fluidic connections are varied across the length of the at least one fluidic connection to improve neurite penetration and producibility.

21. The multi-well plate of claim 1, wherein the length of the at least one fluidic connection is at least 0.25 mm and at the most 2.0 mm.

22. The multi-well plate of claim 1, wherein the aspect ratio of the dimensions of the at least one fluidic connection ranges from 20:1 (W:H) to 1:5 (W:H).

23. The multi-well plate of claim 1, wherein the multi-well plate comprises a 6, 12, 24, 48, 96, 384, 1536 or 3456 well format, and is optionally organized in a 2:3 rectangular matrix.

24. The multi-well plate of claim 1, wherein the multi-well plate comprises at least 2 groups of three neighboring and fluidically interconnected wells.

25. The multi-well plate of claim 1, wherein the multi-well plate comprises at least 3 groups of two neighboring and fluidically interconnected wells.

26. The multi-well plate of claim 1, wherein the multi-well plate comprises at least 1 group of four neighboring and fluidically interconnected wells.

27. A method for high throughput screening of a material of interest, comprising screening the material of interest using the multi-well plate of claim 1.

28. The method of claim 27, wherein the material of interest is a 2D cell culture.

29. The method of claim 27, wherein the material of interest is a 3D cell culture.
